(19) United States
(12) Patent Application Publication
(10) Pub. No.: US 2015/0018820 A1
(43) Pub. Date: Jan. 15, 2015

(54) APPARATUS AND METHODS FOR RENAL DENERVATION

(71) Applicant: BOSTON SCIENTIFIC SCIMED, INC., MAPLE GROVE, MN (US)

(72) Inventors: HONG CAO, MAPLE GROVE, MN (US); HUISUN WANG, MAPLE GROVE, MN (US)

(21) Appl. No.: 14/329,511

(22) Filed: Jul. 11, 2014

Related U.S. Application Data
(60) Provisional application No. 61/845,847, filed on Jul. 12, 2013.

(51) Int. Cl. A61B 18/14

(52) U.S. Cl. CPC A61B 18/1492; A61B 2018/00577
USPC 606/41

(57) ABSTRACT

Some embodiments are directed to medical devices and methods for making and using the medical devices. An exemplary medical device includes a catheter having an elongated shaft and an inflatable balloon mounted at or on a distal portion of the elongated shaft. The catheter further includes a first electrically conductive blade, and a second electrically conductive blade. Each blade may be configured to contact tissue upon inflation of the balloon. The blades may contact the tissue with reduced or minimal incising of the tissue, or even without incising the tissue, within a body lumen. Thermal energy may be applied to the tissue upon electrical energy being applied to the respective blades.

### CROSS-REFERENCE TO RELATED APPLICATIONS

This application claims priority under 35 U.S.C. §119 to U.S. Provisional Application Ser. No. 61/845,847, filed Jul. 12, 2013, the entirety of which is incorporated herein by reference.

### TECHNICAL FIELD

Some embodiments relate to medical devices, such as for renal denervation, and methods for making and using the medical devices. However, other embodiments can have other applications.

### BACKGROUND

A wide variety of intracorporeal medical devices have been developed for medical use, such as for intravascular use. Some of these devices include guidewires, catheters, and/or other apparatus. These devices can be manufactured by any one of a variety of different manufacturing methods, and may be used according to any one of a variety of methods. Each of the related art medical devices and methods is subject to certain advantages and disadvantages. There is an ongoing need to provide alternative medical devices as well as alternative methods for manufacturing and using medical devices.

### SUMMARY

It may therefore be beneficial to provide alternative medical devices as well as methods for manufacturing and using the alternative medical devices. Some embodiments are therefore directed to several alternative designs of medical device structures and assemblies, as well as methods of making and using the alternative medical device structures and assemblies.

Some embodiments are directed to performing perivascular renal nerve tissue ablation. One such illustrative embodiment includes a catheter having an elongated shaft, an inflatable balloon, a first electrically conductive blade, and a second electrically conductive blade. The inflatable balloon is mounted at or on a distal portion of the elongated shaft. The first and second electrically conductive blades are mounted at or on the inflatable balloon, and each blade is configured to contact tissue upon inflation of the balloon within a body lumen. The first and second electrically conductive blades are spaced apart, and contact the tissue with reduced or minimal incising of the tissue, and in some cases without incising the tissue. Subsequently, the electrical energy is applied to the first and second electrically conductive blades to provide thermal energy to the tissue.

Another illustrative embodiment of a catheter includes an elongated shaft, an inflatable balloon, a first pair of electrically conductive blades, and a second pair of electrically conductive blades. The inflatable balloon is mounted at or on a distal portion of the elongated shaft. The first pair of electrically conductive blades serves as a first pair of bipolar electrodes, while mounted at or on the inflatable balloon with a gap therebetween. The first pair of electrically conductive blades is configured to deliver electrical energy sufficient to ablate perivascular renal nerve tissue from within the renal artery. The second pair of electrically conductive blades serves as a second pair of bipolar electrodes configured to deliver electrical energy sufficient to ablate perivascular renal nerve tissue from within the renal artery. The second pair of electrically conductive blades is mounted at or on the inflatable balloon with a gap therebetween.

Yet another illustrative embodiment includes a method of ablating target nerve tissue from a location within a body vessel. The method includes delivering an inflatable balloon, which is mounted at or on a balloon catheter, to a location within the body vessel that is adjacent the target nerve tissue. The balloon catheter includes multiple electrically conductive blades mounted at or on the inflatable balloon. The balloon is inflated at the location within the body vessel to thereby press the electrically conductive blades into contact with a vessel wall of the body vessel. Subsequently, electrical energy is applied to the electrically conductive blades. Thermal energy is applied to the target nerve tissue to ablate this tissue upon electrical energy being applied to the electrically conductive blades.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic view illustrating a renal denervation system in situ;

FIG. 2A is a schematic side view of a catheter for renal denervation;

FIG. 2B is a cross-sectional view taken through line 2B-2B′ in FIG. 2A;

FIG. 3 is a schematic side view of an inflatable balloon configured for coupling to a distal portion of the catheter of FIG. 2A;

FIG. 4 is a schematic side view of another embodiment of the inflatable balloon of FIG. 3;

FIG. 5 is a schematic view illustrating various configurations of electrically conductive blades configured to be used with the inflatable balloons of FIGS. 3 and 4;

FIG. 6A is a schematic side view of another embodiment of the inflatable balloon for use with the medical device of FIG. 2A;

FIG. 6B is a cross-sectional view taken along line 6B-6B′ in FIG. 6A;

FIG. 6C is another cross-sectional view taken along line 6C-6C′ in FIG. 6A;

FIG. 7 is a schematic view illustrating an arrangement of multiple electrically conductive blades at or on the inflatable balloon according to some embodiments of the present disclosure;

FIG. 8 is a schematic view illustrating another arrangement of multiple electrically conductive blades at or on the inflatable balloon according to some embodiments of the present disclosure;

FIG. 9A is a schematic view that illustrates an exemplary method for renal denervation using the medical device of FIG. 2A; and

FIG. 9B is a cross-sectional view taken along line 9B-9B′ in FIG. 9A.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

Definitions of certain terms are provided below and shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same or substantially the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise.

The following detailed description should be read with reference to the drawings, in which similar elements in different drawings are identified with the same reference numbers. The drawings, which are not necessarily to scale, depict illustrative embodiments and are not intended to limit the scope of the disclosure.

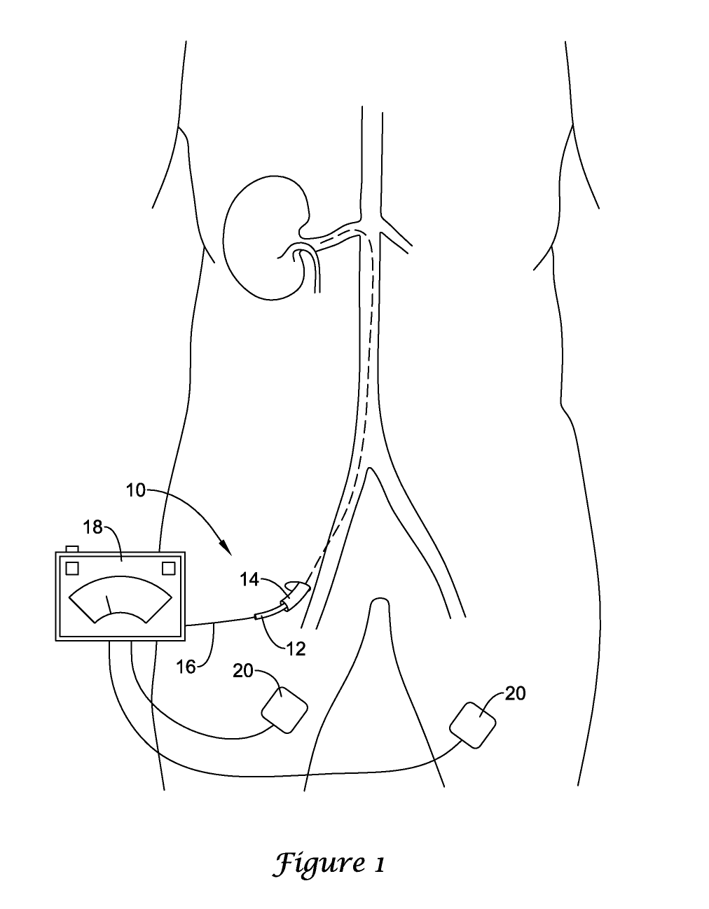

FIG. 1 is a schematic view of an illustrative renal nerve modulation system in situ. A renal ablation system 10 may include one or more conductive element(s) 16 for providing power and a renal nerve modulation device 12, which may optionally be provided within a delivery sheath 14. This structure is shown in more detail in subsequent figures.

A proximal end of conductive element(s) 16 may be connected to a control and power unit 18, which may supply the appropriate electrical energy to activate one or more electrodes disposed at or near a distal end of the renal nerve modulation device 12. The control and power unit 18 may also be utilized to supply/receive the appropriate electrical energy and/or signal to activate one or more sensors disposed at or near a distal end of the renal nerve modulation device 12. If suitably activated, the electrodes are capable of ablating tissue as described below, and the sensors may be used to sense desired physical and/or biological parameters. The terms electrode and electrodes may be considered to be equivalent to element(s) capable of ablating adjacent tissue in the following disclosure. In some embodiments, return electrode patches 20 may be provided at or on a patient's legs or at another location of the patient's body (such as at locations known or otherwise used in the related art) to complete the circuit. The system may also include a proximal hub (not illustrated) having ports for a guidewire, an inflation lumen and/or a return lumen.

The control and power unit 18 may include monitoring elements to monitor parameters, such as power, voltage, pulse size, temperature, force, contact, pressure, impedance, and/or shape, and/or other suitable parameters. Sensors may be mounted along the renal nerve modulation device 12, and suitable controls can be provided for performing a desired procedure. In some embodiments, the control and power unit 18 may control a radiofrequency (RF) electrode, and in turn, may power other electrodes including “virtual electrodes,” which are described herein. The electrode may be configured to operate at a suitable frequency and generate a suitable signal. Other ablation devices may be used as desired, including but not limited to, devices that utilize resistance heating, ultrasound, microwave, and laser technologies. The control and power unit 18 may provide a different form of power to these devices, if desired.

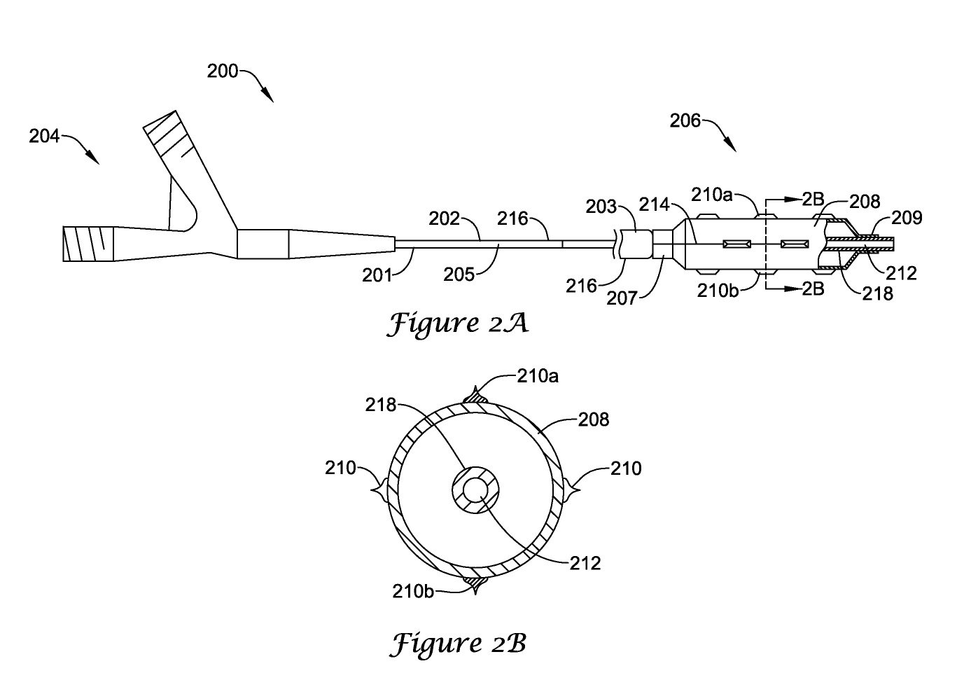

FIG. 2A is a schematic side view of a catheter 200 for renal denervation or other ablation procedures. In the illustrated embodiment, the catheter 200, along with other components, includes an elongated shaft 202, an inflatable balloon 206 coupled at or to a distal portion 203 of the shaft 202, and a plurality of electrically conductive blades, such as electrically conductive blades 210a and 210b, mounted at or on the inflatable balloon 206. Some of the components of the catheter 200 are discussed in detail below.

The elongated shaft 202 may include a tubular member having a proximal portion 201, and one or more lumens extending between the proximal portion 201 and the distal portion 203. The elongated shaft 202 may be configured to have a substantially circular cross-section; however, it may be configured to have other suitable cross-sectional shapes, such as elliptical, oval, polygonal, irregular, etc. In addition, the elongated shaft 202 may be flexible along its entire length, or adapted for flexure only along portions of its length. The required degree of flexibility of the elongated shaft 202 may be predetermined based on its intended navigation to a target vascular passage, and the amount of inertial force required for advancing the elongated shaft 202 through the vascular passage.

The cross-sectional dimensions of the elongated shaft 202 may vary according to the desired application. Generally, the cross-sectional dimensions of the elongated shaft 202 may be sized smaller than the typical blood vessel in which the catheter 200 is to be used, such as in a renal artery. The length of the elongated shaft 202 may vary according to the location of the vascular passage where nerve tissue denervation is to be conducted. In some instances, a 6 F or a 5 F catheter may be used as the elongated shaft 202, where “F,” also known as French catheter scale, is a unit to measure catheter diameter (1 F=⅓ mm). In addition, the elongated shaft 202 or a portion thereof may be selectively steerable. Mechanisms such as, pull wires and/or other actuators may be used to selectively steer the elongated shaft 202, if desired.

The proximal portion 201 of the elongated shaft 202 may include a handle 204 usable to manually maneuver the distal portion 203 of the elongated shaft 202. The handle 204 may include one or more ports that may be used to introduce any suitable medical device, fluid or other interventions. For example, the handle 204 may include a guidewire port in communication with a guidewire lumen 212 (shown in the cut-away portion at the distal end of the catheter 200) which may be used to introduce a guidewire having an appropriate thickness into the elongated shaft 202, which may guide the shaft 202 to the target location within an artery. Furthermore, the handle 204 may include an inflation port configured to be coupled to a source of inflation fluid for delivering an inflation fluid through an inflation lumen of the catheter shaft 202 to the inflatable balloon 206. In certain embodiments, the elongated shaft 202 may one or more additional lumens, which may be configured for a variety of purposes, such as delivering medical devices or for providing fluids, such as saline, to a target location.

The inflatable balloon 206 may be operably coupled at or to the distal portion 203 of the elongated shaft 202. In particular, a proximal portion or waist 207 of the inflatable balloon 206 may be secured to the distal portion 203 of the elongated shaft 202, such as an outer tubular member 216 of the elongated shaft 202. Furthermore, a distal portion or waist 209 of the inflatable balloon 206 may be secured to the distal portion 203 of the elongated shaft 202, such as an inner tubular member 218 of the elongate shaft 202 extending through the outer tubular member 216. Any suitable securing method(s) may be employed to couple the two structures, including but not limited to adhesive bonding, thermal bonding (e.g., hot jaws, laser welding, etc.) or other bonding technique, as desired. The inflatable balloon 206 may be configured to be expanded from a deflated state to an inflated state through delivery of an inflation fluid (e.g., saline) through the inflation lumen of the catheter shaft 202. The balloon 206 may be deflated during introduction of the catheter inside the patient's body, whereas the balloon 206 may be inflated once it reaches the target site within the body vessel.

The inflatable balloon may be manufactured using or otherwise formed of any suitable material, including polymer materials, such as polyamide, polyether block amide (PEBA), polyester, nylon, etc. The inflatable balloon 206 may have a substantially cylindrical configuration with a circular cross-section, as shown in the illustrative embodiment. However, in other embodiments the inflatable balloon 206 may have another suitable configuration or shape, if desired.

The catheter 200 further includes a plurality of electrically conductive blades 210 mounted on the inflatable balloon 206. The electrically conductive blades 210 may be configured to serve as electrodes mounted on the balloon 206. For example, as shown in the cross-section of FIG. 2B, the catheter 200 may include a first electrically conductive blade 210a and a second electrically conductive blade 210b mounted at or on an outer surface 208 of the inflatable balloon 206, as well as additional electrically conductive blades as shown in FIG. 2A, as desired. Each blade 210 may have a longitudinal length of about 3 millimeters, about 4 millimeters, or about 5 millimeters, for example. The plurality of electrically conductive blades 210 may be circumferentially and/or longitudinally spaced from adjacent blades 210 on the inflatable balloon 206. For example, the balloon 206 may include four rows of electrically conductive blades 210 arranged circumferentially around the balloon 206 at about 90° intervals. Additionally or alternatively, each row of electrically conductive blades 210 may include multiple electrically conductive blades 210 longitudinally spaced from adjacent blades 210. In some instances, the blades 210 in one row may be longitudinally offset from the blades 210 in an adjacent row, as shown in FIG. 2A.

In some embodiments, the electrically conductive blades 210 extend radially outwards from the outer surface 208 of the inflatable balloon 206. For example, in some instances, the electrically conductive blades 210 may have a height of about 0.5 millimeters to about 1.0 millimeters. Thus, the radially outwardmost edge or tip of the electrically conductive blades 210 may be located about 0.5 millimeters to about 1.0 millimeters radially outward from the balloon 206. In these embodiments, the electrically conductive blades 210 may be adapted provide enhanced contact with the vessel wall. For example, the blades 210, raised above the outer surface of the balloon 206, may be configured to embed into the surrounding tissue, when the inflatable balloon 206 is in the inflated state within the vessel lumen. In this situation, the embedded electrically conductive blades 210 may form deep thermal lesions in the tissue with focused energy upon application of electrical energy.

In some embodiments, the radially outward projecting edge or tip of the electrically conductive blades 210 may be blunt edges that may reduce or prevent injury to the surrounding tissue (e.g., blunts edges configured to contact the tissue without incising the tissue), when the blades 210 are embedded in the tissue while the balloon 206 is inflated. Alternatively and additionally, the electrically conductive blades 210 may have substantially sharp edges that may facilitate penetrating the electrically conductive blades 210 within the surrounding vessel wall tissue. Blunt edges of the blades 210, such as rounded edges, may provide a surface area that is larger than that provided by sharp edges, and thus the current density may be more uniformly distributed around the edges. As a result, uniform thermal lesions may be created in the surrounding tissue and the depth of the lesion may be increased by pressing the blades 210 against the vessel wall.

Additionally, pressing the blades 210 into the vessel wall may bring the edge of the blades 210 closer to the target tissue (e.g., renal nerves are typically located 2-3 millimeters from the inner surface of the vessel wall).

The electrically conductive blades 210 may be made from or otherwise formed of any suitable electrically conductive material, including but not limited to metals, alloys, polymers, etc. For example, in some instances the electrically conductive blades 210 may be formed of stainless steel, titanium, tungsten, nitinol, or other metallic materials. Any desired number of the electrically conductive blades 210 may be mounted at or on the outer surface 208 of the balloon 206 without departing from the scope of the present disclosure.

In the illustrated embodiment, the electrically conductive blades 210 may have a substantially trapezoidal shape. However, the blades 210 may have any other suitable shape, including but not limited to, rectangular, triangular, serpentine, pyramidal, etc.

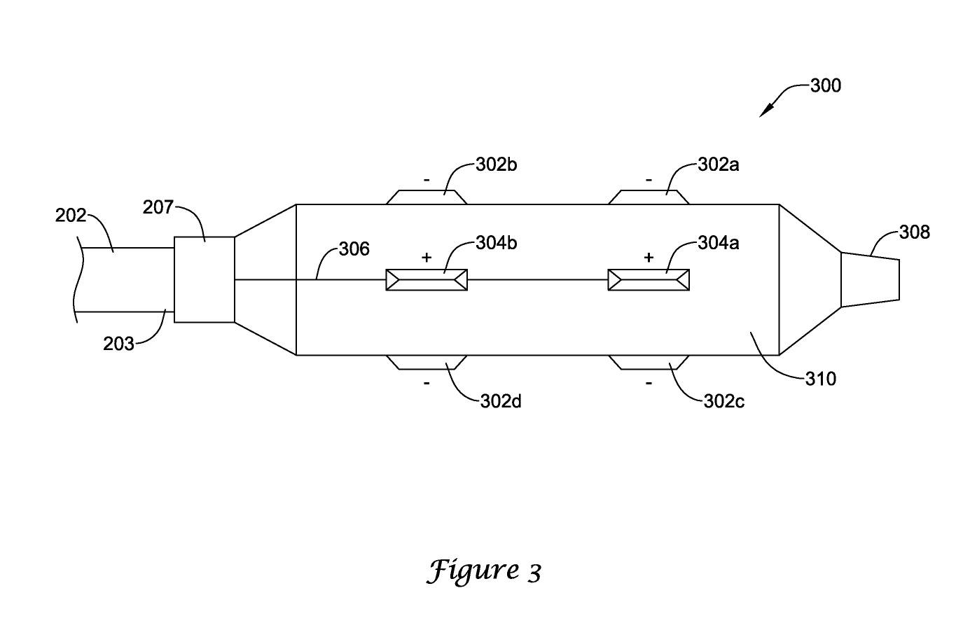

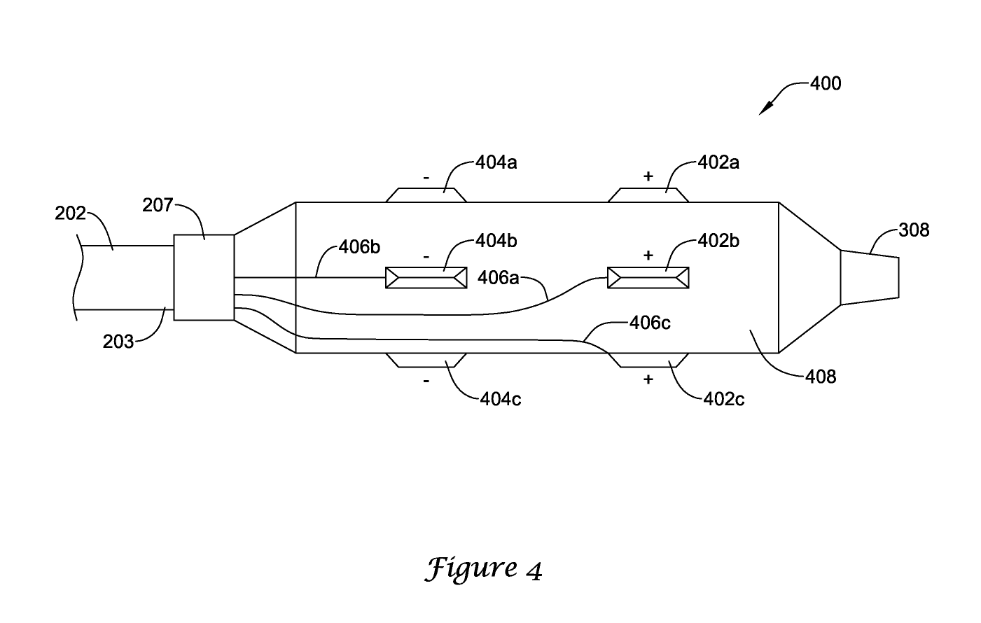

The electrically conductive blades 210 may be spaced apart from one another on the balloon 206 to electrically isolate each electrically conductive blade 210 from adjacent electrically conductive blades 210. According to some embodiments, two electrically conductive blades 210 of opposing polarities may be spaced apart at a distance of equal to or less than 2mm. However, the electrically conductive blades 210 may be spaced apart at any suitable distance as desired. In some embodiments, the spaced apart arrangement of the electrically conductive blades 210 may be employed to form various thermal lesion patterns between a pair of electrically conductive blades 210 electrically coupled in a bipolar arrangement. Some of the lesion patterns may include linear, circumferential, continuous helical, discontinuous helical, etc. These lesion patterns and arrangements of electrically conductive blades 210 are discussed in detail with respect to FIGS. 3 and 4.

The electrically conductive blades 210 that are selected to transmit the electrical energy to form the lesion patterns are activated by passing electrical energy through the electrically conductive blades 210. For example, the catheter 200 may include electrical pathways 214 electrically coupled to the electronically conductive blades 210 for passing electrical energy to/from the electrically conductive blades 210 along (e.g., through) the elongated shaft 202 from an electrical energy source (see FIG. 1). An individual electrical pathway 214 may be electrically coupled to one or more of the electrically conductive blades 210. Thus, the catheter 200 may include one or more, or a plurality of electrical pathways 214, each passing electrical energy to one or more of the electrically conductive blades 210. Electrical energy may be transmitted to the tissue in a monopolar or multipolar (e.g., bipolar, tripolar, etc.) mode, or by any other known, related art, and/or later developed method.

Monopolar mode occurs when the selected one or more of the electrically conductive blades 210 are activated with the same polarity, such as either anode or cathode, with the opposite pole provided as an electrode positioned exterior of the patient (e.g., an electrode patch 20 as shown in FIG. 1). The electrically conductive blades 210 that have the same polarity (i.e., either anode or cathode) may create radially deep lesions around their edges. In the multipolar mode, a pair of the electrically conductive blades 210 may create a thermal lesion between the pair of multipolar electrically conductive blades 210. For example, in the bipolar mode, two electrically conductive blades 210 are activated as anode and cathode, and the electrical energy is transmitted between the selected electrically conductive blades 210. In these situations, the two electrically conductive blades 210 that have opposite polarities should be substantially close to one another. In some embodiments, the distance between the two electrically conductive blades 210 activated in the bipolar mode may be located at a distance of about 1 millimeter apart, about 2 millimeters apart, or about 3 millimeters apart, for example. Although not shown, the catheter 200 may employ one or more sensors, such as temperature sensors to monitor the temperature of the electrically conductive blades 210 and/or the vessel wall. These sensors may be in the form of a thermocouple, thermistor(s), etc. The sensors may be placed at different locations, such as adjacent the electrically conductive blades 210, in order to monitor the temperature of the portions of the blades 210 that are close to the vessel wall to thereby monitor the temperature of the surrounding tissue. As a result, the sensors may reduce or prevent fouling of the electrically conductive blades 210 and over heating of the surrounding tissue.

In some embodiments, the sensors may be configured to provide feedback to the control and power unit 18 (as shown in FIG. 1) for adjustment parameters, including but not limited to, power, voltage, current, duty cycle, duration, etc. In addition, the control and power unit 18 (as shown in FIG. 1) may be configured to raise alerts if any of the sensors detect temperatures over a preconfigured threshold value. If an alert is raised, operators may discontinue modulation until the temperature at the electrically conductive blades 210 and/or at the vessel wall returns under the threshold value. Alternatively, operators may simply monitor the temperatures and discontinue modulation when they feel temperatures exceed a certain value. In some embodiments, impedance may also be measured as an indication of heating and ablation.

FIG. 2B is a cross-sectional view of the inflatable balloon 206 of FIG. 2A taken through line 2B-2B′. As shown, the inflatable balloon 206 may define a circular cross-section surrounding an inner tubular member of the elongated shaft 202, defining the guidewire lumen 212. The guidewire lumen 212 may be adapted to receive a guidewire therethrough for guiding the catheter 200 to a desired treatment location within the vasculature, such as a renal artery, as discussed above. In the illustrated embodiment, the first and the second electrically conductive blades 210a and 210b are mounted at or on the outer surface 208 of the inflatable balloon 206, while being about 180 degrees circumferentially offset to one another. Any number of electrically conductive blades 210 may be arranged and/or mounted at or on the outer surface 208 of the inflatable balloon 206 in any suitable arrangement, which is discussed in more detail below.

FIG. 3 is a schematic side view of an inflatable balloon 300 configured to be used with the catheter 200 of FIG. 2A. The inflatable balloon 300 is similar in shape and structure to that of the inflatable balloon 206 of FIG. 2A. The inflatable balloon 300 includes electrically conductive blades 302a, 302b, 302c, 302d, 304a, and 304b mounted at or on its outer surface 310. In the illustrated embodiment, the electrically conductive blades 302a-d and 304a-b are activated in the bipolar mode, such that the electrically conductive blades 302a-d are cathodes and the electrically conductive blades 304a-b are anodes. Although not shown, the balloon 300 may include additional electrically conductive blades 304 arranged opposite the electrically conductive blades 304a-b, if desired. Hereinafter, the electrically conductive blades 302a, 302b, 302c, and 302d may be referred to as cathodic blades 302 having a negative polarity, whereas the electrically conductive blades 304a and 304b may be referred to as anodic blades 304 having a positive polarity.

In addition, the inflatable balloon 300 has an electrical pathway 306 that may provide electrical energy to the anodic blades 304. The electrical pathway 306 may extend along (e.g., through) the elongated shaft 202. While not shown explicitly, the electrical pathway 306 may travel along the outer surface 310 of the inflatable balloon 300, through the wall of the inflatable balloon 300, or otherwise arranged, such that the electrical pathway 306 may couple to the anodic blades 304 at one end, and to the control and power unit 18 (as shown in FIG. 1) at another end. In the present embodiment, the anodic blades 304 are coupled to a common electrical pathway 306. In an alternate embodiment, however, individual electrically conductive blades 302a-d and 304a-b may be coupled to an individual electrical pathway, as discussed below. Although not shown, the cathodic blades 302 may couple to the control unit through another individual electrical pathway. Alternatively, one pair of cathodic blades 302a and 302b may be electrically coupled to the control unit through a first electrical pathway, whereas another pair of cathodic blades 302c and 302b may be coupled to the control unit through a second electrical pathway independent of the first electrical pathway. In some embodiments, the electrical pathway includes a conductive wire, and each electrical pathway is electrically isolated from one another.

The bipolar blades 302 and 304 are arranged and configured to form a circumferential thermal lesion pattern. To this end, the lesion may be formed between bipolar blade pairs 304a and 302a, 304a and 302c, 304b and 302b, and 304b and 302d. It is noted that additional lesions may be formed between a first anode blade on the opposite side of the balloon 300 and each of blades 302a and 302c, and a second anode blade on the opposite side of the balloon 300 and each of blades 302b and 302d.

The inflatable balloon 300 may further include a distal tip 308 having a blunt edge, which may reduce or avoid tissue injury while navigating the balloon 300 through a body vessel. Therefore, the inflatable balloon 300, having a blunt distal tip 308 and blunt electrically conductive blades 302 and 304, may be atraumatic when used within the patient's body.

FIG. 4 is a schematic side view that illustrates another embodiment of an inflatable balloon 400. The inflatable balloon 400 is similar in structure and shape to that of the inflatable balloon 300 of FIG. 3 and the inflatable balloon 206 of FIG. 2A. The inflatable balloon 400 is shown as including electrically conductive blades 402a, 402b, 402c, 404a, 404b, and 404c mounted at or on an outer surface 408 of the inflatable balloon 400. In the illustrated embodiment, the electrically conductive blades 402a, 402b, and 402c may have anodic (e.g., positive) polarity, and thus be referred to as anodic blades 402 hereinafter. Similarly, the electrically conductive blades 404a, 404b, and 404c may have cathodic (e.g., negative) polarity, and therefore be referred to as cathodic blades 402 hereinafter.

In contrast to the embodiment discussed with respect to FIG. 3 above, the embodiment shown in FIG. 4 may include an individual electrical pathway for each electrically conductive blade. For example, the electrically conductive blade 402b may be coupled to the control unit through an electrical pathway 406a, the electrically conductive blade 404b may be coupled to the control unit through an electrical pathway 406b, and the electrically conductive blade 402c may be coupled to the control unit through an electrical pathway 406c. Although not shown, additional electrical pathways may be provided to the additional electrically conductive blades 402, 404. These electrical pathways 406a-c may extend along the length of the elongated shaft 202 to provide electrical energy to the anodic and cathodic blades 402 and 404. It should be noted that the electrical pathways 406a-c may partially extend along the longitudinal length of the inflatable balloon 400, while travelling either beneath the outer surface 408 or along the outer surface 408, for example.

The anodic and cathodic blades 402 and 404 may be mounted at or on the outer surface 408 so as to form a longitudinal lesion pattern. However, the blades 402 and 404 may be arranged and configured to form any suitable lesion pattern, including but not limited to, helical, circumferential, etc. The longitudinal lesion pattern may be formed as a result of electrically energy passing between the longitudinally aligned pair of bipolar blades. For example, a lesion may be formed between anodic blade 402a and cathodic blade 404a. Similarly, other lesions may be formed between cathodic blade 402b and anodic blade 404b, and cathodic blade 402c and anodic blade 404c.

Although only six blades are shown, any suitable number of blades may be employed for a desired function. For example, an additional bipolar pair of blades 402, 404 may be longitudinally positioned on an opposite side of the balloon 400 from the cathodic blade 402b and anodic blade 404b.

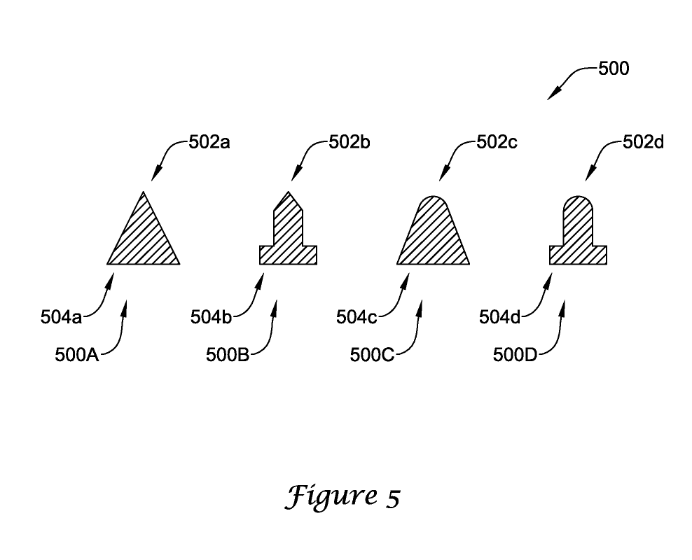

FIG. 5 is a schematic view that illustrates various configurations of electrically conductive blades 500 configured to be used with the inflatable balloons in accordance with this disclosure. As shown, one electrically conductive blade 500A may have a substantially triangular-shaped configuration with a sharp edge or tip 502a extending radially outward from the surface of the balloon for contact with the vessel wall. The blade 500A may have a base 504a configured to be attached at or mounted on the outer surface of the balloon. Another electrically conductive blade 500B may have a T-shaped configuration having a penetrating portion with a substantially sharp edge or tip 502b extending from a base portion 504b. In contrast, electrically conductive blades 500C and 500D are triangular-shaped and T-shaped configurations with bases 504c, 504d for mounting the blades onto the balloon and blunt or rounded tissue contacting edges 502c, 502d, respectively, extending from the bases 504c, 504d and provided as the radially outwardmost portion of the blades 500C, 500D. The tissue contacting edges may extend radially outward from the balloon for contacting the vessel wall upon inflation of the balloon. Since the electrically conductive blades 500C and 500D have blunt or rounded tissue contacting tips or edges 502c, 502d, the blades 500C and 500D may be able to contact and press against the vessel wall without incising the vessel wall. The blade configurations shown in FIG. 5 are illustrative of some possible electrically conductive blades for mounting on the balloon. However, any suitable configuration of electrically conductive blades 500 may be employed without departing from the scope and spirit of the present disclosure.

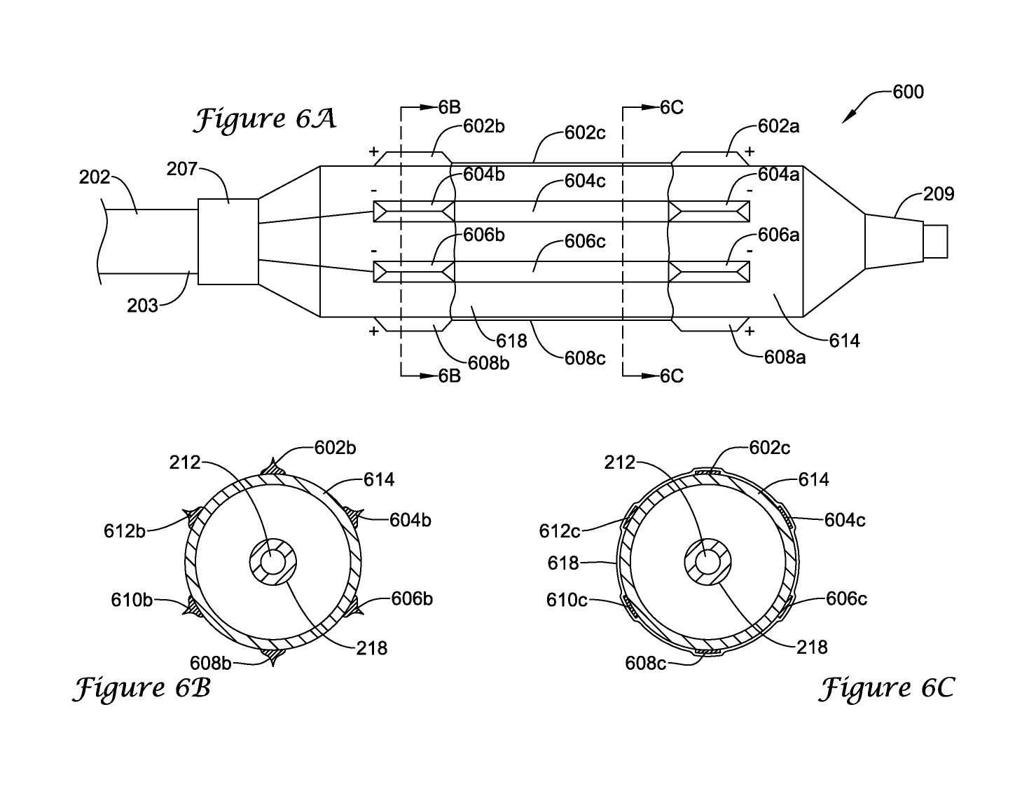

FIG. 6A is a schematic side view of another inflatable balloon 600 for use with the catheter 200 of FIG. 2A. The inflatable balloon 600 may be substantially cylindrically shaped with a circular cross-section, for example. The inflatable balloon 600 may include multiple electrically conductive blades 602, 604, 606, 608, 610 and 612 mounted at or on an outer surface 614 of the inflatable balloon 600. Each electrically conductive blade 602, 604, 606, 608, 610 and 612 may have an elongated-shaped configuration or longitudinal configuration, and be disposed along the longitudinal length of the inflatable balloon 600. In some embodiments, the blades 602, 604, 606, 608, 610 and 612 may only extend along part of the length of the outer surface 614. Alternatively, the blades 602, 604, 606, 608, 610 and 612 may extend along the entire length of the outer surface 614 of the body portion of the balloon 600. Further, each electrically conductive blade 602, 604, 606, 608, 610 and 612 may be formed as a monolithic or unitary member having a plurality of exposed surface portions spaced apart by insulated portions. For example, each electrically conductive blade 602, 604, 606, 608, 610 and 612 may have a first exposed surface, a second exposed surface, and an electrically insulated portion positioned between the first and second exposed surfaces, each of which is discussed in detail below. In such embodiments, the length of the blade may be about 20-25 millimeters, for example, while the length of the exposed portions may be about 3, 4 or 5 millimeters, and the length of the insulated portions may be about 4-15 millimeters, for example.

In some embodiments, a portion of the outer surface 614 may be masked with an insulating member 618 so as to mask or insulate at least a portion of each electrically conductive blade 602, 604, 606, 608, 610 and 612. The insulating material 618 may be wrapped around, or otherwise positioned around a portion of the outer surface 614 in order to divide each electrically conductive blade 602, 604, 606, 608, 610 and 612 into a first exposed surface 602a, 604a, 606a, and 608a (portions of blades 610 and 612 are not shown in FIG. 6A) and a second exposed surface 602b, 604b, 606b, and 606b (portions of blades 610 and 612 are not shown in FIG. 6A), respectively. These first and second exposed surfaces (602a, 604a, 606a, and 608a and 602b, 604b, 606b, and 606b) may act as electrodes and be configured to provide electrical energy to the surrounding tissue when disposed within the blood vessel. Each electrically conductive blade 602, 604, 606, 608, 610 and 612 may have an electrically insulated portion 602c, 604c, 606c, and 608c (portions of blades 610 and 612 are not shown in FIG. 6A) disposed between the first exposed surface 602a, 604a, 606a and 608a and the second exposed surfaces 602b, 604b, 606b, and 606b, respectively. The electrically insulated portion 602c, 604c, 606c, and 608c formed by masking of portion of each blade 602, 604, 606, and 608 may electrically couple the first and second exposed surface portions of the blades 602, 604, 606, 608, 610 and 612 while being electrically insulated from the vessel wall.

In some embodiments, the first and second exposed surface 602a and 602b of the electrically conductive blade 602 may have the same polarity, such as anodic. Similarly, the exposed surfaces 604a and 604b of electrically conductive blade 604 may have cathodic polarity. In such an arrangement, a lesion may be formed between the bipolar pair of blades, such as 602a and 604a, and 602b and 604b, etc. Similarly, the first and second exposed surfaces 606a and 606b of the electrically conductive blade 606 may have cathodic polarity, whereas the first and second exposed surfaces 608a and 608b of the electrically conductive blade 608 may have anodic polarity. Therefore, lesions may be formed between the bipolar pair of blades, such as 606a and 608a, and 606b and 608b, etc. A circumferential lesion pattern may be formed as a result of this structure.

Any electrically insulative material, such as insulative polymers, ceramics, etc., may be employed to form the insulating member. Some embodiments may include a flexible insulative polymeric sleeve that may be wrapped around the outer surface 614. Other embodiments may include a coating as the insulating member 618. Further, the insulating member 618 may be disposed at or on the outer surface 614 to form any suitable lesion pattern. For example, the insulating member 618 may be disposed in a helical arrangement to form a helical lesion pattern. Other suitable arrangements of the insulating member 618 may include longitudinal, circular, etc.

FIG. 6B is a cross-sectional view taken along line 6B-6B′ in FIG. 6A. As shown, the inflatable balloon 600 has a circular cross-sectional shape and includes second exposed surfaces 602b, 604b, 606b, 608b, 610b, and 612b of the electrically conductive blades 602, 604, 606, 608, 610, and 612 disposed at or on its outer surface 614. The electrically conductive blades 602, 604, 606, and 608 may have a substantially triangular cross-section with blunt edges, but can also have other suitable cross-sectional shapes, as desired. The guidewire lumen 212, defined by the inner tubular member of the elongate shaft 202, for example, may extend through the inflatable balloon 600.

FIG. 6C is another cross-sectional view taken along line 6C-6C′ in FIG. 6A. A portion of each blade 602, 604, 606, 608, 610 and 612 is masked or insulated from the vessel wall using the insulating member 618. The masked portion of the blades may form the electrically insulating portions 602c, 604c, 606c, and 608c, as discussed above, electrically insolating the insulating portions form the vessel wall. The insulating portions of the blades 602, 604, 606, 608, 610 and 612 may have a radial height measured from the outer surface of the balloon 600 less than the radial height of the exposed portions of the blades 602, 604, 606, 608, 610 and 612.

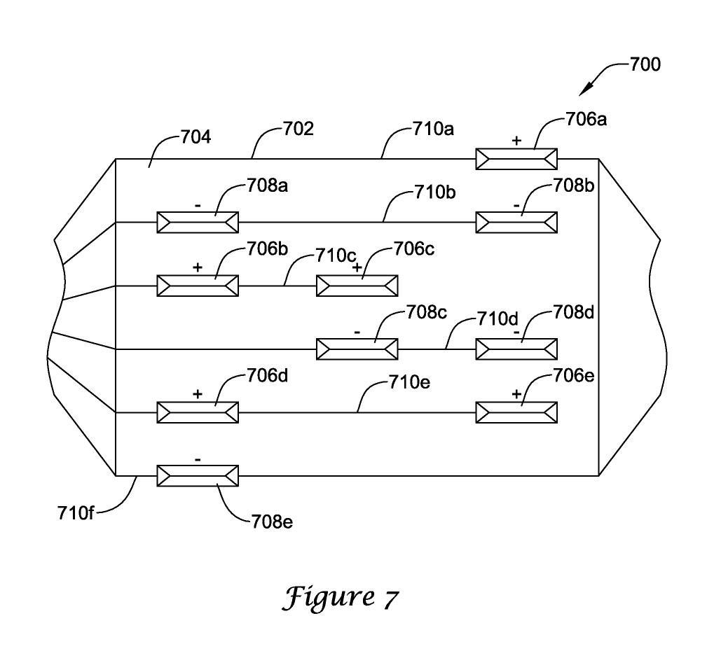

FIG. 7 is a schematic that shows an arrangement 700 of a plurality of electrically conductive blades mounted on an inflatable balloon 702. FIG. 7 illustrates the balloon 702 in a flatten state in which the balloon 702 has been cut lengthwise and laid out flat to illustrate the blade arrangement around the entire circumference of the balloon 702. The inflatable balloon 702 may define an outer surface 704 having a plurality of electrically conductive blades 706a-e and 708a-e disposed thereon. The electrically conductive blades 706a-e are anodic (e.g., positive electrodes), whereas the electrically conductive blades 708a-e are cathodic (e.g. negative electrodes). The electrically conductive blades aligned along the longitudinal length of the outer surface 704 may be coupled through a common electrical pathway, such as a conductive wire, as discussed above. For example, the electrically conductive blade 706a is coupled to an electrical pathway 710a, whereas the electrical conductive blades 708a and 708b are coupled through another electrical pathway 710b. Similarly, blades 706b and 706c are coupled to an electrical pathway 710c, blades 708c and 708d are coupled to an electrical pathway 710d, blades 706d and 706e are coupled to an electrical pathway 710e, and blade 708e is coupled to an electrical pathway 710f. Each electrical pathway 710a-f may be isolated from one another, and electrical energy may be delivered to respective electrical conductive blades through the electrical pathways coupled thereto. The electrical pathways may extend to the handle assembly of the catheter to be coupled to a source of electrical energy.

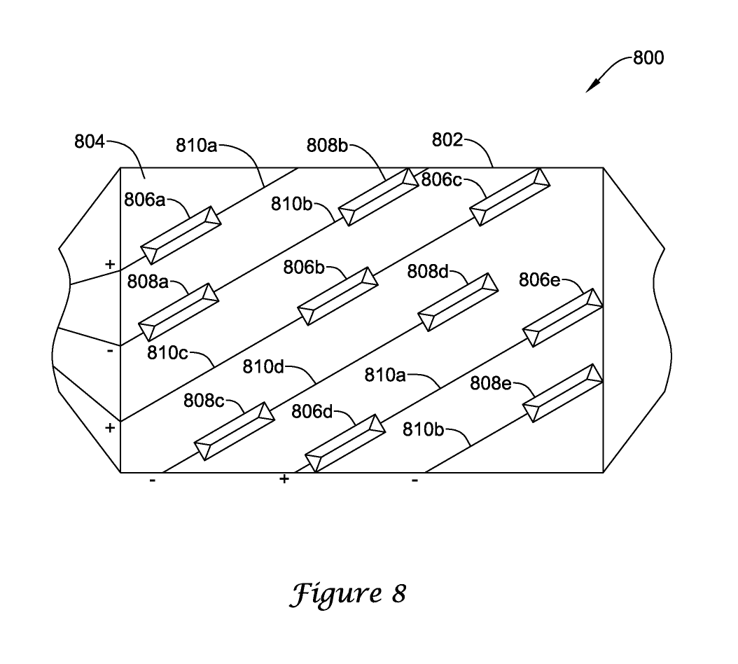

The bipolar electrically conductive blades 706a-e and 708a-e may be arranged so as to form a discontinuous helical thermal lesion pattern. However, the bipolar electrically conductive blades 706a-e and 708a-e may be arranged so as to form other suitable lesion patterns, including but not limited to, circumferential, longitudinal, irregular, etc. One such arrangement is shown with respect to FIG. 8, which is discussed below.

Electrical energy may be provided, such as selectively provided, to one or more of the electrical pathways to send electrical energy to the corresponding electrically conductive blade(s). Electrical energy may pass between electrically conductive blades of opposing polarity to generate a thermal lesion in the vessel wall therebetween.

FIG. 8 is a schematic that shows another arrangement 800 of a plurality of electrically conductive blades mounted on an inflatable balloon 802. Similar to the embodiment shown in FIG. 7, FIG. 8 illustrates the inflatable balloon 802 in a flattened state by cutting the balloon lengthwise along its longitudinal axis to lay the balloon out flat to illustrate the blade arrangement around the entire circumference of the balloon 802.

The inflatable balloon 802 may define an outer surface 804 having a plurality of electrically conductive blades 806a-e and 808a-e disposed thereon. The electrically conductive blades 806a-e are anodic (e.g., positive electrodes), whereas the electrically conductive blades 808a-e are cathodic (e.g., negative electrodes). The electrically conductive blades aligned along the longitudinal length of the outer surface 804 may be coupled through a common electrical pathway, such as a conductive wire, as discussed above. To this end, the electrical conductive blade 806a is coupled to an electrical pathway 810a, whereas the electrical conductive blades 808a and 808b are coupled through another electrical pathway 810b. Similarly, blades 806b and 806c are coupled to an electrical pathway 810c, blades 808c and 808d are coupled to an electrical pathway 810d, blades 806d and 806e are coupled to an electrical pathway 810e, and blade 808e is coupled to an electrical pathway 810f. Each electrical pathway 810a-f may be isolated from one another, and electrical energy may be delivered to respective electrical conductive blades through the electrical pathways coupled thereto. The electrical pathways may extend to the handle assembly of the catheter to be coupled to a source of electrical energy.

The bipolar electrically conductive blades 806a-e and 808a-e may be arranged so as to form a discontinuous helical thermal lesion pattern. However, the bipolar electrically conductive blades 706a-e and 708a-e may be arranged so as to form other suitable lesion patterns, including but not limited to, circumferential, longitudinal, irregular, etc.

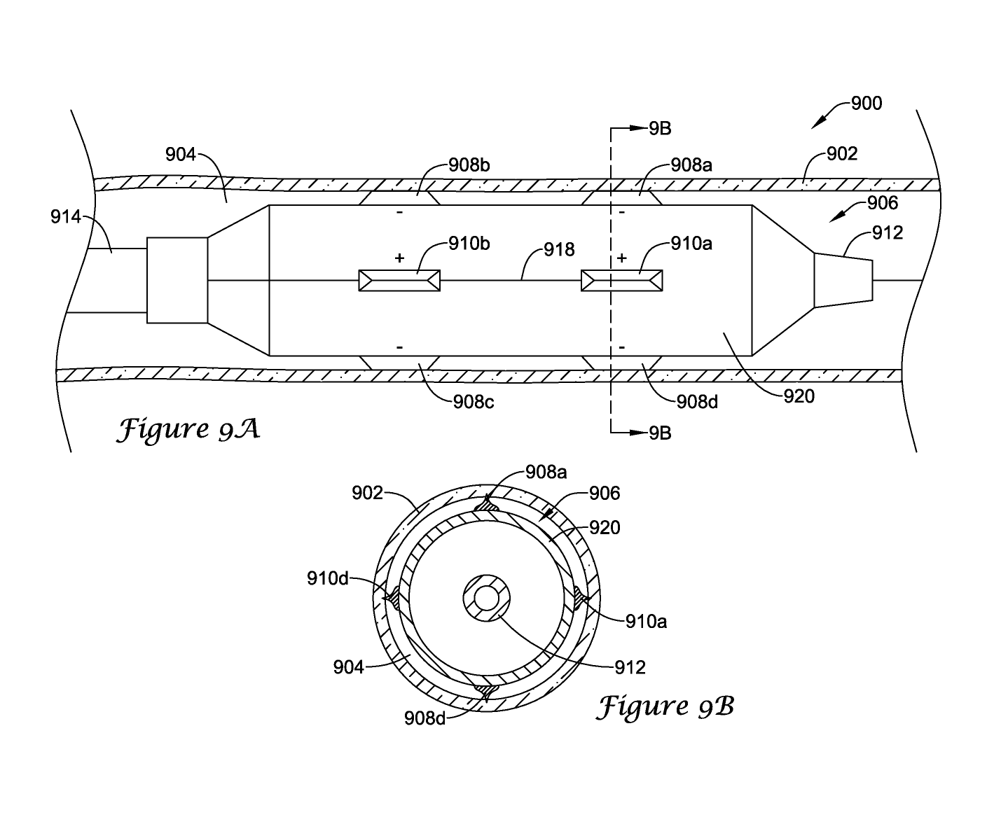

FIG. 9A illustrates an exemplary method of ablating target nerve tissue from a location within a vessel 902 of a patient's body. For example, the illustrated method may be utilized to perform perivascular renal nerve tissue ablation from within the lumen of a renal artery. A medical device 900 may be disposed within a vessel lumen 904 of the vessel 902. The medical device 900 may include an elongated shaft 914 having an inflatable balloon 906 mounted on a distal portion thereof The elongated shaft 914 may have the same shape and structure as the elongated shaft 202 shown in FIG. 2A, for example. The inflatable balloon 906 may have a similar structure and function as that of the inflatable balloon 206 shown in FIG. 2A. However, the inflatable balloon 906 may be configured similar to one or more other embodiments described herein, or otherwise configured with a plurality of electrically conductive blades mounted thereon.

The method may include introducing the medical device 900 to a target location (as shown in FIG. 9A, for example) within the body vessel 902. For instance, the medical device (e.g., balloon catheter) may be advanced over a guidewire to the target location, such as a location within a renal artery. During delivery, the inflatable balloon 906 may be in the deflated state, as discussed above. A distal tip 912 of the inflatable balloon 906 may be rounded to reduce or avoid injury to the vessel during introduction. The balloon 906 may be inflated to the inflated state once the medical device 900 is navigated to reach the target within the vessel 902. In the inflated state, multiple electrically conductive blades 908a-d and 910a-b that are mounted at or on an outer surface 920 of the inflatable balloon 906 may be pressed against, embed within, or otherwise contact the wall of the body vessel 902. In some instances, the electrically conductive blades 908a-d and 910a-b may be embedded into the vessel wall without incising the vessel wall. For example, the tip or outwardmost edge of the electrically conductive blades may be pressed against or into the vessel wall to position the electrically conductive blades closer to the nerve tissue to be ablated (which may be positioned proximate the outer surface of the vessel wall). Because the electrically conductive blades extend radially outward from the balloon 906, precise sizing of the balloon to match the diameter of the vessel lumen may be alleviated, and oversizing of the balloon 906 may be unnecessary.

Once the electrically conductive blades 908a-d and 910a-b contact the vessel wall of the body vessel 902, electrical energy may be applied to the electrically conductive blades. The electrical energy may be carried through one or more electrical pathways, such as an electrical pathway 918. The electrical pathway may deliver electrical energy to the electrical conductive blades 910a and 910b, which are anodic by polarity. Although not shown, a common electrical pathway may be employed to deliver electrical energy to the cathodic set of electrically conductive blades 908a and 908b, and/or the cathodic set of electrically conductive blades 908c and 908d. Sufficient electrical energy provided to the blades 908a-d and 910a-b may apply thermal energy to the target nerve tissue to thermally ablate the target nerve tissue.

In present embodiment, the four bipolar sets of blades 908a-d and 910a-b may form a circumferential lesion pattern; however, any suitable pair of blades may be employed to form any lesion pattern, such as a discontinuous helical lesion pattern, a continuous helical lesion pattern, a discontinuous circumferential lesion pattern, or a longitudinal lesion pattern, or other lesion pattern, as discussed above.

FIG. 9B is a cross-sectional view taken along line 9B-9B′ in FIG. 9A. The inflatable balloon 906 may be disposed within the vessel lumen 904 in the inflated state, such that the electrically conductive blades 908a, 910a, 908d, and 910d contact the vessel wall. A gap may be provided between the contacting electrically conductive blades 908a, 910a, 908d, and 910d and the vessel wall, which may ensure continuous blood flow within the vessel 902 while the balloon 906 is inflated. The blood flow may provide sufficient cooling within the vessel lumen 904 to reduce or avoid damage of the blades, excessive heating at the surface of the vessel wall, and/or fouling of the blood. Further, the edges or tips of the electrically conductive blades 908a, 910a, 908d, and 910d in engagement with the vessel wall may be blunt and/or rounded to reduce or avoid injury to the tissue during contact (e.g., to avoid incising the tissue of the vessel wall). In addition, the blunt surface of the electrically conductive blades 908a, 910a, 908d, and 910d may provide a greater surface area, which may provide a uniform or substantially uniform current density distribution upon application of electrically energy.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one exemplary embodiment in other embodiments. The disclosure's scope is, of course, defined in the language in which the appended claims are expressed.

What is claimed is:

1. A catheter comprising:
an elongated shaft;
an inflatable balloon mounted at a distal portion of the elongated shaft;
a first electrically conductive blade mounted at the inflatable balloon, the first electrically conductive blade configured to contact tissue without incising the tissue upon inflation of the balloon within a body lumen and apply thermal energy to the tissue when electrical energy is applied to the first electrically conductive blade; and
a second electrically conductive blade mounted at the inflatable balloon and spaced from the first electrically conductive blade, the second electrically conductive blade configured to contact tissue without incising the tissue upon inflation of the balloon within a body lumen and apply thermal energy to the tissue when electrical energy is applied to the second electrically conductive blade.

2. The catheter of claim 1, wherein the first and second electrically conductive blades each includes a blunt edge configured to contact the tissue without incising the tissue.

3. The catheter of claim 2, wherein the first and second electrically conductive blades are configured to deliver electrical energy sufficient to ablate perivascular renal nerve tissue from within a renal artery.

4. The catheter of claim 1, further comprising a first electrical pathway extending along the elongated shaft to provide electrical energy to the first electrically conductive blade.

5. The catheter of claim 4, further comprising a second electrical pathway extending along the elongated shaft to provide electrical energy to the second electrically conductive blade.

6. The catheter of claim 5, wherein the first electrical pathway provides electrical energy to the first electrically conductive blade independent of the second electrical pathway providing electrical energy to the second conductive blade.

7. The catheter of claim 1, wherein the first electrically conductive blade includes a first exposed portion serving as a first electrode of a first polarity and a second exposed portion serving as a second electrode of a first polarity, wherein the first electrically conductive blade includes an electrically insulated portion between the first exposed portion and the second exposed portion.

8. The catheter of claim 7, wherein the second electrically conductive blade includes a first exposed portion serving as a first electrode of a second polarity and a second exposed portion serving as a second electrode of a second polarity, wherein the second electrically conductive blade includes an electrically insulated portion between the first exposed portion and the second exposed portion, the second polarity being opposite the first polarity.

9. The catheter of claim 8, wherein when the first and second electrically conductive blades are contacting tissue, an electrical pathway passes through the tissue between the first electrode of the first electrically conductive blade and the first electrode of the second electrically conductive blade, and a second electrical pathway passes through the tissue between the second electrode of the first electrically conductive blade and the second electrode of the second electrically conductive blade.

10. A catheter comprising:
an elongated shaft;
an inflatable balloon mounted at a distal portion of the elongated shaft;
a first pair of electrically conductive blades mounted at the inflatable balloon with a gap therebetween, the first pair of electrically conductive blades serving as a first pair of bipolar electrodes configured to deliver electrical energy sufficient to ablate perivascular renal nerve tissue from within the renal artery; and
a second pair of electrically conductive blades mounted at the inflatable balloon with a gap therebetween, the second pair of electrically conductive blades serving as a second pair of bipolar electrodes configured to deliver electrical energy sufficient to ablate perivascular renal nerve tissue from within the renal artery.

11. The catheter of claim 10, wherein the first pair of electrically conductive blades are spaced axially and circumferentially from the second pair of electrically conductive blades such that a thermal lesion formed by the first pair of bipolar electrodes is offset axially and circumferentially from a thermal lesion formed by the second pair of bipolar electrodes.

12. The catheter of claim 10, wherein the first and second pairs of electrically conductive blades each have a blunt edge configured to contact the tissue without incising the tissue.

13. The catheter of claim 10, wherein the gap between the first pair of electrically conductive blades is about 2 millimeters or less, and the gap between the second pair of electrically conductive blades is about 2 millimeters or less.

14. The catheter of claim 10, further comprising:
a first temperature sensor positioned proximate the first pair of electrically conductive blades; and
a second temperature sensor positioned proximate the second pair of electrically conductive blades.

15. The catheter of claim 10, wherein the first and second pairs of electrically conductive blades project radially outward from an outer surface of the balloon such that the first and second pairs of electrically conductive blades embed into a vessel wall of the renal artery upon inflation of the balloon.

16. The catheter of claim 10, further comprising:
a third pair of electrically conductive blades mounted on the inflatable balloon with a gap therebetween, the third pair of electrically conductive blades serving as a third pair of bipolar electrodes configured to deliver electrical energy sufficient to ablate perivascular renal nerve tissue from within the renal artery; and
a fourth pair of electrically conductive blades mounted on the inflatable balloon with a gap therebetween, the fourth pair of electrically conductive blades serving as a fourth pair of bipolar electrodes configured to deliver electrical energy sufficient to ablate perivascular renal nerve tissue from within the renal artery.

17. The catheter of claim 16, wherein the first, second, third and fourth pairs of bipolar electrodes are configured to create a helical lesion pattern within the renal artery.

18. A method of ablating target nerve tissue from a location within a body vessel, comprising:
delivering an inflatable balloon of a balloon catheter to a location within the body vessel adjacent the target nerve tissue, the balloon catheter including a plurality of electrically conductive blades mounted at the inflatable balloon;
inflating the balloon at the location within the body vessel to press the plurality of electrically conductive blades into contact with a vessel wall of the body vessel;
applying electrical energy to the plurality of electrically conductive blades; and
applying thermal energy to the target nerve tissue to ablate the target nerve tissue when electrical energy is applied to the plurality of electrically conductive blades.

19. The method of claim 18, wherein applying thermal energy to the target nerve tissue ablates perivascular nerves of a renal artery in a substantially helical pattern.

20. The method of claim 18, wherein the plurality of electrically conductive blades are arranged on the balloon to create a helical lesion pattern on the vessel wall without incising the vessel wall.
